(19) United States
(12) Patent Application Publication
(10) Pub. No.: US 2015/0272916 A1
(43) Pub. Date: Oct. 1, 2015

(54) MEDICAMENT COMPRISING AT LEAST ONE OMEGA-3 POLYUNSATURATED FATTY ACID

(71) Applicant: CENTRE HOSPITALIER UNIVERSITAIRE VAUDOIS, Lausanne (CH)

(72) Inventors: Heidi Schuster, Giessen (DE); Ewald Schlotzer, Oberursel (DE); Ulrich Suchner, Erding (DE); Mette Berger, St.-Légier (CH); Luc Tappy, Cugy (CH); Lucas Liaudet, Lausanne (CH); Rene Chiolero, Grimisuat (CH)

(21) Appl. No.: 14/440,470

(22) PCT Filed: Nov. 6, 2013
(86) PCT No.: PCT/EP2013/073105

§ 371 Date: May 4, 2015

(30) Foreign Application Priority Data
Nov. 7, 2012 (EP) 12191656.3

(51) Int. Cl. A61K 31/202

(52) U.S. Cl. CPC A61K 31/202

(57) ABSTRACT

A medicament comprising a therapeutically effective amount of at least one omega-3 polyunsaturated fatty acid for use in the reduction and prevention of post-ischemic damages in patients undergoing elective surgery.

### BACKGROUND OF THE INVENTION

The present invention is directed to a medicament for use in the treatment of patient undergoing an ischemic event. The medicament described is in particular useful for the treatment of patients undergoing elective surgery, in particular elective heart surgery.

Omega-3 polyunsaturated fatty acids (also referred to n-3 polyunsaturated fatty acids or omega-3 fatty acids) have many benefits and one of them (alpha-linolenic acid) is considered as an essential fatty acid, meaning that it cannot be synthesized by the human body but is vital for normal metabolism. Common dietary sources of omega-3 polyunsaturated fatty acids include fish oil and algal oil. Heidt M C, et al. in Thorac Cardiovasc Surg. 2009; 57:276-80 “Beneficial effects of intravenously administered N-3 fatty acids for the prevention of atrial fibrillation after coronary artery bypass surgery: a prospective randomized study” for example describes the pre-operative use of Omegaven (fatty acid emulsion produced by Fresenius Kabi) in the reduction of atrial fibrillation. WO2007/059431A1 relates to a method of limiting neurological damage resulting from hypoxic ischemia comprising administering an omega 3 lipid emulsion after the cerebral hypoxic ischemia insult. U.S. Pat. No. 5,053,387 “omega-3 fatty acids in traumatic injury treatment” (Wesley Alexander J) describes that Omega-3 fatty acids in combination with other nutrients can be used for treatment of traumatic injury to improve immunologic response and reduce hypermetabolic response.

Patients undergoing surgery may suffer from ischemic insult and its influence on organ function. Accordingly, there is a need for an improved medication for treating such patients.

### SUMMARY OF THE INVENTION

The present invention generally relates to a medicament comprising a therapeutically effective amount of at least one omega-3 polyunsaturated fatty acid for use in the reduction and prevention of post-ischemic damages in patients undergoing elective surgery. For example for use in reduction and prevention of post-ischemic damages associated with reperfusion injuries.

One exemplary advantage of such an administration is the reduction of time until the patient can be weaned of machine assisted mechanical ventilation compared to a group of patients in a placebo group. Further advantages will become evident throughout the detailed description.

Thus, an exemplary embodiment refers to a medicament comprising a therapeutically effective amount of at least one omega-3 polyunsaturated fatty acid for use in reducing the machine assisted mechanical ventilation time of patients recovering from surgery induced ischemia, such as ischemia induced by elective surgery.

Further advantageous embodiments of the invention are defined in the dependent claims and will become apparent from the detailed description.

### DETAILED DESCRIPTION

“Major surgery” as used herein refers to surgery upon the chest or abdomen which typically involves a risk to a patient's life, require general anesthesia, and typically at least 24-48 hours of intensive care therapy for postoperative stabilisation of the vital functions. A major surgery may be a surgery selected from the group consisting of cardiovascular surgery, vascular bypass surgery, cardiopulmonary bypass techniques (e.g. coronary artery bypass graft (CABG) surgery with or without associated valvular surgery, abdominal/thoracic aorta aneurysm surgery, major abdominal aortic procedures) and solid organ transplantation.

“Elective surgery” as used herein refers to surgery that is scheduled more than 24 hours in advance. An elective surgery may be a surgery selected from the group consisting of major surgeries specified above, i.e. cardiovascular surgery, vascular bypass surgery, cardiopulmonary bypass techniques (e.g. coronary artery bypass graft (CABO) surgery with or without associated valvular surgery, abdominal/thoracic aorta aneurysm surgery, major abdominal aortic procedures) and solid organ transplantation.

“Urgent surgery” as used herein is surgery that may wait until the patient is medically stable, but needs performed within less than 24 hours after diagnosis (e.g. progressive inferior limb ischemia caused by thrombosis of an aortic aneurysm).

“Emergency surgery” is a surgery that must be performed without delay. Practically, emergency surgery will be performed as soon as a surgeon is available. (e.g. dissection of thoracic aorta, ruptured abdominal aortic aneurysm, complications of percutaneous transluminal coronary angioplasty (PTCA) procedure such as ruptured coronary artery requiring immediate surgery).

“Mechanical ventilation” as used herein refers to a method to mechanically assist or replace spontaneous breathing. Mechanical ventilation includes manual mechanical ventilation and machine assisted mechanical ventilation.

“Manual mechanical ventilation” refers to breathing assisted by a physician, respiratory therapist or any other suitable person compressing a bag or set of bellows.

“Machine assisted mechanical ventilation” as used herein refers to a method to assist or replace spontaneous breathing involving a machine (ventilator). The term includes invasive ventilation and non-invasive ventilation. Machine assisted mechanical ventilation may be achieved by: positive pressure ventilation, where air (or another appropriate gas mix) is pushed into the trachea, and negative pressure ventilation, where air is essentially sucked into the lungs.

“SOFA-Score” as used herein refers to the Sequential Organ Failure Assessment score. It is used to track a patient's status by quantification of the magnitude and of the evolution of organ failures during the stay in an intensive care unit (ICU). The SOFA score is a validated scoring system to determine the extent of 6 independent organ functions or their rate of failure. The total score results from the sum of the six organ scores, one each for the respiratory, cardiovascular, hepatic, coagulation, renal and neurological systems. Each organ is rated from 0 “no failure” to 4 which represent the worst possible organ failure.

The following scoring tables are used:

Respiratory System

| PaO2/FiO2 (mmHg) | SOFA score |
|---|---|
| <400 | 1 |
| <300 | 2 |
| <200 and mechanically | 3 |
| ventilated | |
| <100 and mechanically | 4 |
| ventilated | |

Nervous System

| Glasgow coma scale | SOFA score |
|---|---|
| 13-14 | 1 |
| 10-12 | 2 |
| 6-9 | 3 |
| <6 | 4 |

Cardio Vascular System

| Mean Arterial Pressure OR administration | |
|---|---|
| of vasopressors required (vasopressor | |
| drug doses are in mcg/kg/min) | SOFA score |
| MAP < 70 mm/Hg | 1 |
| dop <= 5 or dob (any dose) | 2 |
| dop > 5 OR epi <= 0.1 OR nor <=0.1 | 3 |
| dop > 15 OR epi > 0.1 OR nor >0.1 | 4 |

Liver

| Bilirubin (mg/dl) [μmol/L] | SOFA score |
|---|---|
| 1.2-1.9 [>20.5-32.5] | 1 |
| 2.0-5.9 [34.2-100.9] | 2 |
| 6.0-11.9 [102.6-203] | 3 |
| >12.0 [>205] | 4 |

Coagulation

| Platelets × 103/mcl | SOFA score |
|---|---|
| <150 | 1 |
| <100 | 2 |
| <50 | 3 |
| <20 | 4 |

Renal System

| Creatinine (mg/dl) [μmol/L] | |
|---|---|
| (or urine output) | SOFA score |
| 1.2-1.9 [110-170] | 1 |
| 2.0-3.4 [171-299] | 2 |
| 3.5-4.9 [300-440] (or <500 ml/d) | 3 |
| >5.0 [>440] (or <200 ml/d) | 4 |

In cases where the physiological parameters do not match any values of the tables above, zero points are given. In cases where the physiological parameters match values associated with more than one row, the row with most points is picked.

“Mean SOFA score” as used herein is calculated as the mean of scores of the patients on one single day.

“Peak airway pressure” as used herein is measured at the airway opening (Pao) and is a parameter routinely displayed by typical mechanical ventilators for hospital use. It represents the total inspiratory pressure needed to push a predefined volume of gas into the lung. This pressure is the sum of the pressure resulting from inspiratory flow resistance (resistive pressure), the pressure resulting from the elastic recoil of the lung and chest wall (elastic pressure), and the pressure resulting from the alveolar pressure present at the beginning of the breath (positive end-expiratory pressure [PEEP]):

Thus:

Peak airway pressure=resistive pressure+elastic pressure+PEEP

Peak airway pressures vary normally between 10 and 20 cmH2O

FIG. 1 illustrates the components of the airway pressure during the mechanical ventilation, illustrated by an inspiratory-hold manoeuvre.

“Pre-operative administration” refers to administration before surgery, preferably, the medicament herein is administered at least pre-operatively at least the day before surgery.

“Peri-operative administration” as used herein is to be understood as including at least two different administrations relative to the time span wherein surgery is conducted. For example, before and after surgery, before and during surgery (also referred to as intra-operatively), during and after surgery. Other examples will become apparent form the below. Herein, peri-operative administration typically represents a combination from at least two administrations selected from the group consisting of “before and after surgery” and “before and during surgery”. In preferred embodiments peri-operative administration represents an administration before, during and after surgery.

“Ischemia” or ischemic event generally refers to any type of interrupted blood flow which leads to an undersupply of oxygen to the tissue. Ischemia may cause tissue injury directly, or in association with reperfusion. Ischemia, if not reversed in due time, ultimately leads to necrosis. Moreover, restoration of blood flow after a period of ischemia causes additional damage (i.e. reperfusion injury) by oxidative damage (liberation of free radicals generated during the period of ischemia and the reflow) and the liberation of cytokines. Reperfusion and in particular injuries therefrom, influences the outcome after e.g. myocardial infarction, solid organ transplantation, and cardiovascular surgery.

“Medicament” refers to a medicinal product which is intended for use in the cure, treatment or prevention of disease. It is not intended for nutritional purpose and preferably does not contain amino acids or proteins and carbohydrates.

“Substantial surgery induced ischemia” refers to an inevitable ischemic event caused by surgery. In particular, surgery induced ischemia refers to ischemia which is consciously planned and necessary for performing the surgery. In such cases, ischemia is typically induced by intentionally intermitting blood flow to certain body parts or organs for a specific amount of time by clamping arteries.

If not stated otherwise, relative terms used herein in context of describing the recovery of patients, such as faster recovery, faster weaning of mechanical ventilation, lower peak airway pressure, lower variability etc. generally refer to a measurable difference when compared to a placebo group.

The inventors found that surprisingly positive physiological effects can be achieved by the peri-operative administration of omega-3-polyunsaturated fatty acids. The effect was supported by the addition of alpha-tocopherol.

The inventors found that peri-operative administration of the medicament according to the present disclosure improves patient recovery after elective surgery, in particular after major elective surgery such as cardiovascular surgery, vascular bypass surgery, cardiopulmonary bypass techniques (e.g. coronary artery bypass graft (CABG) surgery with or without associated valvular surgery, abdominal/thoracic aorta aneurysm surgery, major abdominal aortic procedures) and solid organ transplantation.

An improved recovery can typically be seen in shorter recovery times reflected by certain physiological parameters in comparison to placebo groups. In particular, positive changes of several different physiological parameters reflect better recovery.

For example, the inventors found that a patient's respiratory system may recover faster, leading to the faster weaning of machine assisted mechanical ventilation. A particularly positive influence has been found in a faster reduction of the peak airway pressure of patients.

Also, a patient's inner organs may over all recover faster which can be seen in lower SOFA scores compared to placebo groups.

Further, the inventors found that peri-operative administration of the medicament according to the present disclosure stabilizes physiological parameters of a patient and thereby contributes to lowering the physiological stress experienced by the patient. Lower variability has been observed in the blood glucose level, blood lactate level, body temperature and heart rate curve of a patient. Also, a lower body temperature has been observed.

As stated above, the invention relates to a medicament comprising a therapeutically effective amount of at least one omega-3 polyunsaturated fatty acid for use in the reduction and prevention of post-ischemic damages in patients, such as patients experiencing surgery induced ischemia, for example ischemia induced by elective surgery. Such a surgery may require a substantial induction of ischemia. Exemplary surgeries are selected from the group consisting of cardiac surgery requiring extracorporeal bypass circulation (e.g. coronary artery bypass graft surgery with or without associated valvular surgery), other cardiac surgeries requiring cardiopulmonary bypass and solid organ transplantation.

The medicament will typically be administered intravenously. Accordingly, it may be formulated to be suitable for intravenous injection/infusion. The medicament of the present disclosure may typically be used for reduction and prevention of post-ischemic damages in patients, wherein the use is further characterized by one or more of the following

a) inducing a lower peak airway pressure in a patient;
b) improving ventilation of a patient;
c) lowering the variability of blood glucose level and/or lowering the variability in lactate level and/or lowering the variability in body temperature and/or lowering the variability in heart rate of a patient, and/or lowering the body temperature of a patient;
d) decreasing the sequential organ failure assessment (SOFA) score of a patient, which results in the additional prevention and/or treatment of
a) Postoperative respiratory dysfunction,
b) Metabolic instability such as postoperative impairment of glucose homeostasis,
c) Postoperative febrile response related tot he post surgical stress response, and/or
d) Postoperative organ dysfunctions as summarized in the SOFA score which most likely occur due to ischemia-reperfusion injury following major elective surgery.

The improvement of ventilation of a patient can be a reduction in time wherein machine assisted mechanical ventilation is necessary

In some embodiments, the medicament of the present disclosure may be used for reduction and prevention of post-ischemic damages in patients, wherein the use is further characterized by at least two, three or all physiological parameters selected from the group consisting of lowering the variability of blood glucose level, lowering the variability in lactate level, lowering the variability in body temperature and lowering the variability in heart rate of a patient; preferably wherein the use is characterized by at least two, three or all physiological parameters selected from the group consisting of lowering the variability of blood glucose level, lowering the variability in lactate level and decreasing the SOFA score of a patient.

In one exemplary embodiment, the medicament comprising a therapeutically effective amount of at least one omega-3 polyunsaturated fatty acid for use in the reduction and prevention of post-ischemic damages in patients undergoing elective surgery and experiencing substantial surgery induced ischemia in an elective surgery, wherein the use is further characterized by the improvement of ventilation of a patient such as for a reduction in time wherein machine assisted mechanical ventilation is necessary.

In preferred embodiments the medicament is administered peri-operatively. In such embodiments, the medicament may preferably be administered at least on the day before surgery in combination with at least on one or both of
on the day of surgery (i.e. at pre-medication and/or during surgery)
the day after surgery.

In one embodiment the medicament is administered at least on the day before surgery in combination with the day after surgery. In on embodiment, the medicament is administered at least on the day before surgery in combination with at pre-medication and on the day after surgery. Administration before surgery may for example be started at least 3 days before, at least 2 days before or at least the day before surgery.

Administration after surgery may for example be continued for at least the day after surgery, at least 2 days after surgery or at least 3 days after surgery.

In one embodiment, the administration is on the evening before, pre-operatively (at pre-medication) and/or intra-operatively (during surgery) and immediately after surgery.

In some embodiments, even though less preferred, the medicament may be administered at least pre-operatively.

In preferred embodiments, the medicament will be administered
a) at least on one of the day before surgery and/or pre-operatively (at pre-medication)
b) optionally intra-operatively (during surgery)
c) and immediately after surgery and/or the day after surgery.

For example, particularly good results can be achieved when the administration of the medicament is the day before surgery, such as on the evening before, and on the day of surgery (at pre-medication and/or during surgery) and immediately after surgery.

Generally, it may be beneficial to administer the medicament for at least on 3 days in a row. For example, the medicament may be administered at least 5 days in a row, or at least 4 days in a row. Administration should preferably include at least the day before surgery, the day of surgery and the day after surgery.

The medicament may be administered peri-operatively as described in any of the embodiments above in one or more doses of 0.01 to 0.5. g, or 0.05-0.3 g, or 0.15-0.25 g omega-3 polyunsaturated fatty acid/kg body weight, such as in one to five doses, for example in three doses. In some embodiments, one to three doses of 0.01 to 0.5. g, or 0.05-0.3 g, or 0.15-0.25 g omega-3 polyunsaturated fatty acid/kg body weight may be administered. For example, two doses of about 0.1 gram omega-3-polyunsaturated fatty acid/kg body weight are given pre-operatively and one dose of about 0.1 gram omega-3-polyunsaturated fatty acid/kg body weight is given post-operatively.

Each of the described doses may be administered over a time period of about 10 min-6 hours, such as over 30 min to 5 hours, preferably 1 hour-4 hours, for example 1.5 hours to 2.5 hours.

In some embodiments, the medicament comprises eicosapentaenoic acid (EPA) and/or docosahexaenoic acid (DHA).

In one preferred embodiment, the omega-3 polyunsaturated fatty acids, such as DHA and EPA, of the medicament are provided by fish oil.

Accordingly, in one embodiment the medicament may be administered in one or more doses of 0.01 to 0.5. g fish oil/kg body weight, for example in one to five doses or three doses. Such a medicament may be administered, at least pre-operatively, and preferably peri-operatively according to any of the embodiments described above.

While in principle, any omega-3 polyunsaturated fatty acid can be used for the medicament according to the present invention, it is preferred that the omega-3 polyunsaturated fatty acid is at least one of hexadecatrienoic acid (HTA), a-linolenic acid (ALA), stearidonic acid (SDA), eicosatrienoic acid (ETE), eicosatetraenoic acid (ETA), eicosapentaenoic acid (EPA), heineicosapentaenoic acid (HPA), docosapentaenoic acid (DPA), docosahexaenoic acid (DHA), tetracosapentaenoic acid and/or tetracosahexaenoic acid.

Preferably, the composition further contains arachidonic acid, in particular 0.9-4.7 g/l arachidonic acid.

It is further preferred that the composition further contains linoleic acid, in particular 1.8-9.0 g/l linoleic acid.

In a preferred embodiment, the composition further comprises alpha-tocopherol, in particular 150-300 mg/l alpha-tocopherol.

In a preferred embodiment, the medicament according to the present invention comprises Omegaven.

Omegaven is a fatty acid emulsion produced by Fresenius Kabi. The contents of Omegaven in grams in 100 ml emulsion correspond to

| | |
|---|---|
| Highly refined fish oil containing: | 10.0 g |
| Eicosapentaenoic acid (EPA) | 1.25-2.82 g |
| Docosahexaenoic acid (DHA) | 1.44-3.09 g |
| dl-α-tocopherol (antioxidant) | 0.015-0.0296 g |

In one exemplary embodiment, such a medicament for use in the reduction and prevention of post-ischemic damages in patients undergoing elective surgery may be administered as described above, wherein the use is further characterized by improving ventilation of a patient. Preferably, such a medicament comprises eicosapentaenoic acid (EPA) and/or docosahexaenoic acid (DHA), e.g. provided by fish-oil. The administration may comprise the doses described above and the medicament will be administered on the day before surgery in combination with at least on one or both of
on the day of surgery (i.e. at pre-medication and/or during surgery)
the day after surgery.

In a further exemplary embodiment, such a medicament for use in the reduction and prevention of post-ischemic damages in patients undergoing elective surgery may be administered as described above, wherein the use is further characterized by decreasing the SOFA-score of a patient. Preferably, such a medicament comprises eicosapentaenoic acid (EPA) and/or docosahexaenoic acid (DHA), e.g. provided by fish-oil. The administration may comprise the doses described above and the medicament will be administered on the day before surgery in combination with at least on one or both of
on the day of surgery (i.e. at pre-medication and/or during surgery)
the day after surgery.

In a further exemplary embodiment, such a medicament for use in the reduction and prevention of post-ischemic damages in patients undergoing elective surgery may be administered as described above, wherein the use is further characterized by lowering the peak-airway pressure of a patient. Preferably, such a medicament comprises eicosapentaenoic acid (EPA) and/or docosahexaenoic acid (DHA), e.g. provided by fish-oil. The administration may comprise the doses described above and the medicament will be administered on the day before surgery in combination with at least on one or both of
on the day of surgery (i.e. at pre-medication and/or during surgery)
the day after surgery.

### Study

In order to evaluate the effect of the medicament according to the present invention for use in the treatment of an ischemic condition in a patient, a study has been conducted which is described in the following.

### Patients

Consecutive patients referred to an institution for elective cardiac surgery (coronary artery bypass graft (CABG) surgery) were screened on the day before surgery. The inclusion criteria were: 18>age<85 years, coronary artery bypass graft with or without valve surgery on cardiopulmonary bypass, normal sinus rhythm. Exclusion criteria were: absence of consent, participation in another trial, emergency or heart beating surgery, current use of antiarrhythmic medications, uncontrolled dyslipidemia, acute or chronic renal failure (plasma creatinine>150 micromole/l), liver cirrhosis (Child A and up), coagulopathy, fish consumption more than twice weekly, treatment with steroids and allergy to fish.

Study design. The study was designed as a prospective, randomized, blinded, placebo controlled trial: patients and care givers were blinded to the intervention, while the research team, not involved in patient care, was unblinded during the intervention. The assessors were blinded for statistical outwork.

Eligible patients were assigned to one of the two study arms according to a computer-generated randomization list: 1) fish oil (FO) infusions plus usual care; and 2) placebo infusion plus usual care. Sequence generation was based on a block size of four, using sequentially numbered, sealed, envelopes. The intervention consisted of either an intravenous lipid emulsion infusion with 0.2 g/kg FO (Omegaven®, Fresenius Kabi A G, Stanz, Switzerland, which mainly contains EPA and DHA) or saline (both hidden in a black plastic bag), administered 3 times: on the evening prior to surgery, before surgery, and on post-operative day 1. The solutions (153 ml lipid or saline infusion as a mean) were infused via a peripheral venous canula over 3 hours.

Anaesthetic, surgical and post-operative management were provided according to standard protocols. Midline sternotomy and standard surgical techniques for cardiopulmonary bypass and CABG were used. Myocardial protection was afforded with cold potassium cardioplegia. No prophylactic steroids were administered. Propofol was avoided for anaesthesia and post-operative sedation. A piece of atrial auricle was obtained during cardiac canulation. The tissue sample (containing essentially cardiomyocytes, but also endothelial cells and fibroblasts) was processed as indicated below for platelets. Once in the ICU, the patients were ventilated using Adaptive Support Ventilation adjusted to ideal body weight followed by Intermittent Positive Support, and were extubated according to our standard protocol (2).

### Study Endoints

The primary endpoint was incorporation of EPA and DHA into the membrane of circulating platelets and atrial tissue cells. The secondary endpoints were cardiovascular and major organ function, inflammatory response to surgery, safety data (peri-operative bleeding, transfusion requirements) and clinical outcome (severity scores, length of machine assisted mechanical ventilation ICU and hospital stay).

### Patient Variables

Age, weight, BMI and pre-operative cardiac disease severity (Euroscore (3)) were recorded. After ICU admission the physiological alterations were assessed with the APACHE II score, and daily sequential organ failure assessment (SOFA) score (4) until post-operative day 5. The blood glucose was targeted at 5-8 mmol/L using insulin infusion by means of a nurse driven protocol blood glucose (5). The 24 h insulin requirements were recorded.

### Blood Determinations

Seven blood samples (15 ml) were collected from the evening before surgery (Day-1=T0), before and after each infusion of FO or placebo, until the morning after surgery (Day 1=T7) for various laboratory determinations.

### Measurements

After ICU admission, arterial blood samples were drawn every 1-2 hours for blood glucose determination.

Plasma triglycerides and non esterified fatty acids (NEFAs): blood was collected in heparin-lithium containing tubes and centrifugated (1420 G for 20 min at 4° C.) to extract plasma for triglycerides and NEFAs measurements. Enzymatic determination of triglycerides was performed with TG PAP 150 kit (BioMerieux, Lyon, France). NEFAs concentrations were measured with a colorimetric method, using a kit from Wako (Neuss, Germany).

### Membrane Fatty Acid (FA) Composition

Blood drawn in EDTA containing syringes was kept on melting ice for a maximum of one hour before processing. Erythrocytes were eliminated by a low speed centrifugation (228 g for 10 min at 4° C.), the supernatant was submitted to a high speed centrifugation (1428 g for 20 min). The platelet-containing pellet was washed by two successive cycles of centrifugationresuspension in TRIS buffer (1000 g for 10 min). Platelets were transferred into conservative containing tubes and stored at −80° C. until analysis. The triglycerides, phospholipids and cholesterylesters contained in the platelets were first separated by a two-dimension thin layer chromatography, then phospholipids FA were analyzed by gas chromatography (Agilent, GC system, 6890 series (6890A) (1). Fatty acid composition is reported as molar percent of total FA composing the phospholipids of the platelets membrane (100*mole of particular FA/mole of total FA). The atrial biopsy was trimmed to discard macroscopic fat, cut into small pieces, homogenized, immediately frozen in dry ice, and further processed as the platelets.

### Inflammatory Markers

Plasma C-reactive protein (CRP by standard method), IL-6, IL-8 and IL-10 were determined twice (baseline, day1) using Biolegend panel kit and the Luminex method on a Bio-plex apparatus (Bio-Rad, www.bio29FEB-8rad.com/BioPlexSystem) by the core facility of the Center for Integrative Genomics, University of Lausanne, Switzerland.

### Hemodynamics and Organ Function

The follow-up of cardiovascular status included mean arterial pressure, vasopressor requirement (total 24 hr norepinephrine dose), heart rate and rhythm analysis (continuous ECG-Holter recording for 24 hours after surgery), as well as total doses of anti-arrhythmic drugs. Core temperature and arterial pressure were retrieved at 10 min intervals from our computerized information system (MetaVision, iMDsoft, Tel Aviv). Renal status was evaluated by daily plasma creatinine. Gas exchange was evaluated by the PaO2/FiO2 ratio on ICU admission and until extubation. The SOFA score was calculated until post-operative day 5, including the intermediate care stay (6). Blood losses during the first 24 hours were recorded.

### Glucose Metabolism

Blood glucose was determined using blood gas analysis every 1-2 hours during the first post-operative day, and insulin dose to maintain a 5-8 mmol/l target were recorded (total 24 hr insulin dose extracted from the computer system).

Endogenous glucose production, total glucose utilization and glucose clearance rate were measured on day 1 using a primed (2 mg/kg) continuous infusion of 6.6 2H2-glucose (Cambridge Isotope Laboratory, Cambridge, Mass., USA). Blood samples were taken at 30 min intervals for determination of plasma 6.6 2H2-glucose and calculation of total glucose turnover using Steele's equations as described (7).

### Statistical Analysis

Patient characteristics, outcome variables, cytokines, triglycerides, NEFAs and SOFA score are expressed as mean±SD. Membrane EPA and DHA content is expressed as median and interquartile range. Two-way ANOVA was used to analyze cytokine changes. Comparison of quantitative variables was performed with unpaired Student t test (pooled 10 minute temperature, HR, and MAP values) and Wilcoxon rank-sum test (glucose, lactate and HbCO). Statistical package: STATA 11.2 StataCorp, College Station, Tex., USA.

### Results of the Study Conducted

Altogether 31 patients were enrolled of which only 28 could be analyzed (Table 1). Surgery was cancelled in one FO patient, and another FO patient retracted his consent. One placebo patient suffering a Steinert myotonic dystrophy had a prolonged ICU stay related to respiratory failure caused by the primary disease, which was not listed among the original exclusion criteria. He was therefore qualified as <<erroneous inclusion>> and secondarily excluded. Baseline characteristics of the 2 groups were similar. At 24 hours after ICU admission, severity scores tended to be lower in patients assigned to FO (p=0.058 for APACHEII). We did not detect any clinically significant side effect related to FO, specifically no increase in post-operative bleeding and in requirements of red blood cell transfusions. There was no hospital death.

### Peak Airway Pressure Significantly Decreased Following Fish Oil in Fusion.

FIG. 3 shows a comparison of peak airway pressure in the FO group and the placebo group.
FO group 16.2 mmHG
Placebo group: 19.0 mmHG
P value between groups p<0.0001

### Impact on Length of Machine Assisted Mechanical Ventilation in Hours

The length of the machine assisted mechanical ventilation could be markedly reduced following FO infusion.
FO group: 9.0±3.3 h
Placebo group: 20±24.3 h
P value between groups: p=0.102

### Impact on Lipid Concentrations and Membrane Contents

Plasma triglycerides concentrations increased markedly following each FO infusion. This effect was transient, as indicated by the return of plasma triglycerides to normal before the following infusion (FIG. 2). Plasma NEFA remained within normal ranges without any significant difference between groups at any time (FIG. 2).

The platelet and cardiac tissue incorporation of n-3 PUFAs is shown in FIG. 3. In platelets, the basal content of EPA was low, amounting less than 0.5% of the total FA composition. Administration of FO promoted a highly significant progressive incorporation of EPA, which was more pronounced after each infusion. In contrast, the basal DHA content reached approximately 2% of the total lipid composition, with no further incorporation after FO.

Incorporation of n-3 PUFAs in atrial tissue was evaluated only once after two of the three doses of FO, given that atrial biopsies were obtained during surgery. In a way similar to platelets, the basal content of EPA was low and it significantly increased after FO. The basal DHA content was higher than in platelets (amounting approximately 6% of the total lipid content), and it did not increase after the infusion of FO.

Effects on CPB-induced systemic inflammation core temperature during the first 24 h after surgery disclosed significantly different time-courses in placebo and FO-treated patients (FIGS. 4 & 4a). Rank test analysis of all 10 min values of the first 24 hours showed a highly significant difference between group (p<0.0001). After the initial phase of re-warming (4 hours), temperature remained lower in the FO group. The interleukins Il-6 and Il-8 (FIG. 8) disclosed significant increases after CPB in all patients (26). FO was associated with a significant attenuation of the IL-6 increase, as well as with a marginal reduction of IL-8 increase (p=0.07). CRP did not differ between groups increasing on day 1 to 75±10 mg/l and 69±10 mg/l in FO and P respectively. Carboxyhemoglobin can be considered a biomarker of inflammation and oxidative stress (2,3): it was significantly lower during the first post-operative day in the FO patients.

### Effects on Cardiac Rhythm and Hemodynamics

There was no significant change in the hemodynamic profile during the first post-operative day (FIGS. 5 and 6), heart rate was much more uniform in the FO group (FIG. 5a). There was no significant impact on arrhythmias which were rare in the cohort: there were only 2 patients with atrial fibrillation (1 per group), 9 with isolated premature ventricular complexes (3 in FO and 6 in placebo), 4 with supraventricular tachycardia (2 per group), 2 requiring post-operative temporary pacing for bradycardia (2 per group) (ns). Combinations of arrhythmias were observed in some patients with no difference between groups.

The mean SOFA score was lower during the 5 days in the FO group (FIG. 7). Length of machine assisted mechanical ventilation was 11 hours shorter in the FO group, the difference remaining a trend. There was no significant impact on the PaO2/FiO2 ratios. Kidney function did not differ as reflected by plasma creatinine and BUN. Length of ICU stay was 16 hours shorter in the FO group.

### Effects on Glucose Metabolism

The average glycaemia during the first 24 h after CPB as well as the variability of the glucose levels was significantly lower with FO (FIG. 8. Insulin requirements during the first 24 hours did not differ (54±19 U/d versus 64±41 U/d in FO and placebo group respectively).

On day 1, endogenous glucose production was similar in both groups (1.94±0.36 in FO versus 1.94±0.28 mg/kg/min in placebo), with a trend to a faster mean clearance rate in the FO group (0.0021±0.0006 versus 0.0017±0.0002 ml/kg/min; p=0.11).

### Discussion of the Study Results

### Main Findings

Two pre-operative infusions of 0.2 g/kg n-3 PUFAs in patients undergoing on pump CABG resulted in a significant increase of the EPA content of platelets and atrial tissue within 12 hours of first administration. This EPA platelet membrane incorporation was further enhanced by a 3rd post-operative infusion. To our knowledge this is the first study showing a nearly immediate incorporation of n-3 PUFA into cardiac tissue after intravenous lipid infusions. This membrane composition modification was associated with a clinically relevant modulation of the inflammatory responses following CPB.

N-3 PUFAs modulate the inflammatory processes by several mechanisms, particularly by inhibiting the production of pro-inflammatory cytokines and the expression of adhesion molecules and of numerous genes involved in inflammation (11). Some of these effects may be mediated through incorporation of PUFAs in plasma membranes leading to alterations of lipid raft structure and function, and membrane trafficking (12). The demonstration of a rapid incorporation of the n-3 PUFA into platelets and cardiac tissue is therefore of particular interest, as it implies the feasibility of a simple intervention to achieve a nearly immediate response (6).

When searching for possible effects mediated by n-3 PUFA, it was observed that the temperature remained lower in the FO group for 24 hours while the increase of IL-6 concentration, a prototypical biomarker of the systemic inflammatory response triggered by cardiopulmonary bypass (8), was significantly attenuated, consistent with a reduced overall inflammatory response.

HbCO was measured, which is considered to be an indirect indicator of endogenous CO production related to the heme-oxygenase induction (13) and a biomarker of systemic inflammation and oxidative stress (9), notably following CPB (10). The significantly lower post-operative levels of HbCO in the FO patients may be considered an argument in favor of a reduced activity of heme-oxygenase and subsequent reduction of the vasodilator carbon monoxide (14).

Cardiovascular effects, organ function and clinical course after CPB Heart rate was modestly higher in the FO group during the first 12 hours after surgery. The first post-operative 24 hour arrhythmia rate did not differ. Without whishing to be bound by theory, the absence of effect may be due to the overall low rate of arrhythmia in this group, precluding the assessment of antiarrytmic effects of FO, or to a low therapeutic effect related to an insufficient FO dose or duration of infusion. It should be underlined that the ability of FO to prevent arrhythmia remains controversial. A recent meta-analysis showed no significant effects of FO supplementation on atrial arrhythmia prevention in cardioversion and cardiac surgery populations (15). Another meta-analysis of controlled human studies suggests that n-3 PUFAs directly or indirectly affect cardiac electrophysiology (16).

Calo et al showed that 2 g of PUFA taken for at least 5 days before CABG substantially reduced the incidence of post-operative atrial fibrillation (54.4%) and was associated with a shorter hospital stay (17).

Despite identical pre-operative characteristics (age and Euroscores), we observed smoother post-operative course with attenuation of ICU alterations in the FO patients as suggest by the lower APACHEII, SOFA scores and machine assisted mechanical ventilation time. As the intervention was started the evening before ICU admission, the severity scores, assessed 24 hours after ICU admission represent the effect of the pre-operative FO modulation, and not a baseline difference between the 2 populations.

### Impact on Glucose Metabolism

Lower blood glucose values were observed in the FO group, and were associated with lower lactate values. But there was no significant impact on either endogenous glucose production, glucose clearance, or daily insulin requirements. This effect on glucose and lactate concentrations nonetheless suggests a beneficial effect on stress-induced insulin resistance and on tissue dysoxia, most likely mediated, again, by anti-inflammatory effects of FO.

### CONCLUSION

A clinically relevant improvement during the early post-operative course, characterized by shorter machine assisted mechanical ventilation times, attenuated inflammatory responses and lower severity scores (SOFA), smoothening of curves of vitality parameters such as hart rate, glucose level, temperature has been observed. Moreover a generally lower body temperature in the fish-oil group has been observed.

TABLE 1
Patient characteristics and clinical outcome variables with numbers
required to reach significance according to power calculation

| | Fish oil | Placebo | P value | LSN |
|---|---|---|---|---|
| N | 14 | 14 | | |
| Gender (males/females) | 14/0 | 11/3 | 0.066 | |
| Age, yrs | 64.7 ± 10.5 [64] | 66.3 ± 9.5 [67] | 0.681 | |
| Weight, kg | 76.3 ± 8.1 [76.5] | 79.8 ± 16.3 [78.8] | 0.477 | |
| BMI, kg/m² | 26.44 ± 2.43 [28.5] | 29.25 ± 5.18 [29.4] | 0.077 | 34.155 |
| Euroscore | 5.1 ± 2.2 [5] | 4.7 ± 2.1 [4] | 0.666 | |
| CABG/CABG + valve, n | 11/3 | 12/2 | 0.621 | |
| Duration of CPB, min | 95 ± 33 | 93 ± 35 | 0.808 | |
| APACHE II score | 13.9 ± 3.6 [14.5] | 16.1 ± 2.1 [16.0] | 0.058 | 29.654 |
| SOFA of first 24 hrs | 6.1 ± 1.3 [6.5] | 6.3 ± 1.5 (6.0] | 0.691 | |
| Bleeding of first 24 hrs, ml | 873 ± 450 [745] | 1030 ± 1032 [770] | 0.606 | |
| Packed red blood cell | 64 ± 128 [0] | 381 ± 724 [0] | 0.118 | 43.834 |
| transfusion, ml | | | | |
| Norephinephrine dose, | 3.37 ± 3.08 | 7.12 ± 12.01 | 0.268 | 86.454 |
| mg/24 hrs | | | | |
| Insulin dose, U/24 hrs | 53.2 ± 19.3 | 63.4 ± 40.4 | 0.399 | 149.279 |
| Mechanical ventilation, hrs | 9.0 ± 3.3 [9.7] | 20.1 ± 24.3 [12.5] | 0.10 | 39.985 |
| Length of ICU stay, hrs | 34.5 ± 18.0 [25.5] | 50.7 ± 32.9 [46.0] | 0.118 | 43.795 |
| Length of hospital stay, d | 12.7 ± 4.2 [11] | 12.2 ± 4.3 [11] | 0.758 | |

Values are means ± SD and median
CABG = coronary artery bypass graft,
CPB = cardiopulmonary bypass,
LSN Least significant number (with α = 0.05)

### DESCRIPTION OF THE FIGURES

FIG. 1: Illustrates the components of the airway pressure during the machine assisted mechanical ventilation, illustrated by an inspiratory-hold maneuver (18).

FIG. 2: Time-course of plasma triglyceride and NEFAs concentrations. A transient rise of triglycerides was followed by a rapid return to pre-infusion values after each FO infusion, while NEFA did not change significantly with the infusions.

FIG. 3: EPA and DHA incorporation into platelets and in cardiac tissue: While EPA increased progressively in platelets, DHA membrane content was unchanged (T0=baseline, T12=after 2nd infusion, T24=after 3^{rd} infusion). EPA is significantly higher in cardiac tissue, while DHA is not.

FIG. 4: Time-course of core temperature (10 min intervals) present over time or as the means of all values.

FIG. 4a: Mean value of all 10 minute values over the first 24 postoperative hours. The Wilcoxon test (P<0.0001) refers to this analysis (ns by 2 way ANOVA)

FIG. 5: Time-course of heart rate in ICU (10 min intervals): the box plot presentation reflects the lower spreading of the heart rate values in the FO group

FIG. 6: Time-course of mean arterial pressure (10 min intervals).

FIG. 7: Time-course of the SOFA scores.

FIG. 8: Arterial glucose concentration in the 2 groups showing significantly lower values in the FO group during 24 hours after surgery.

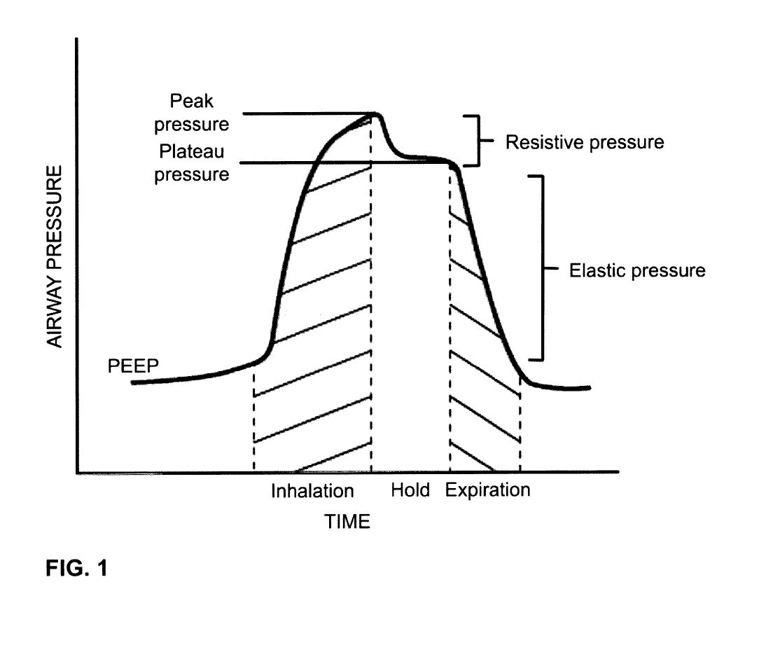

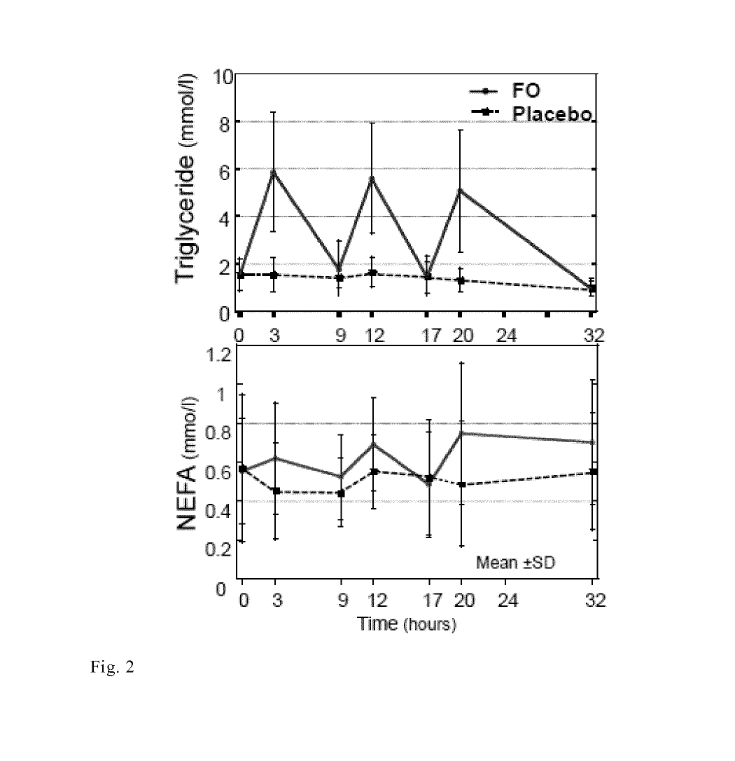

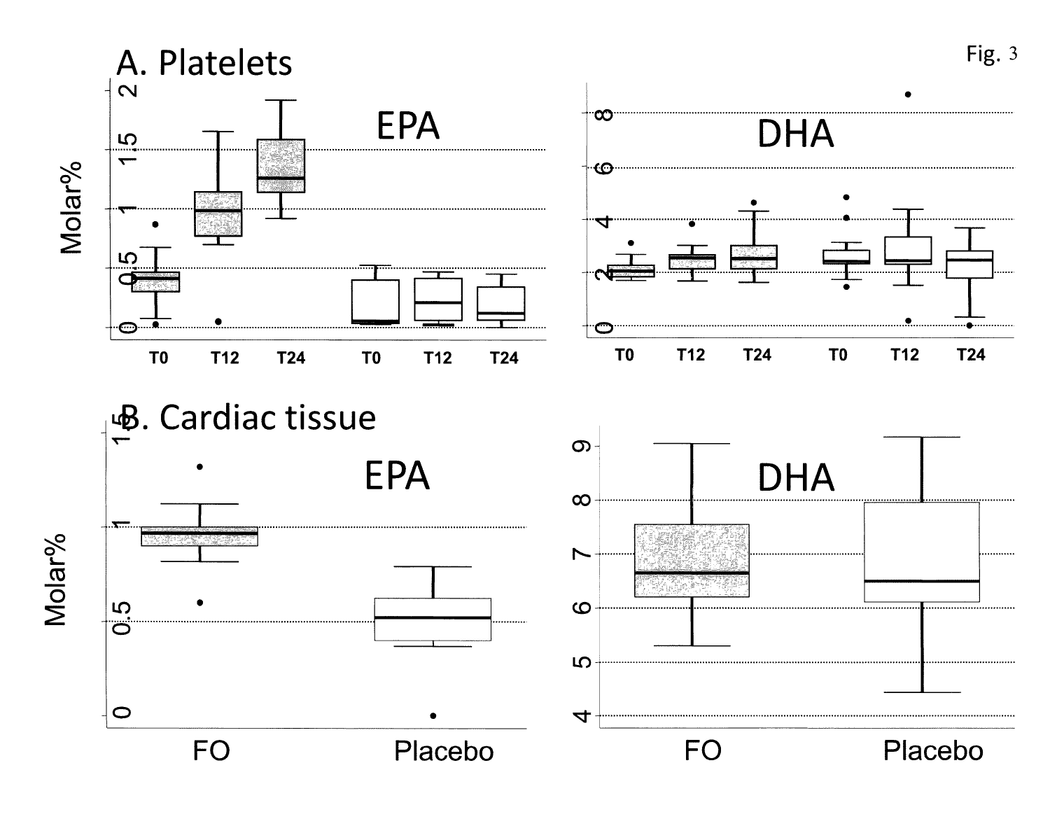

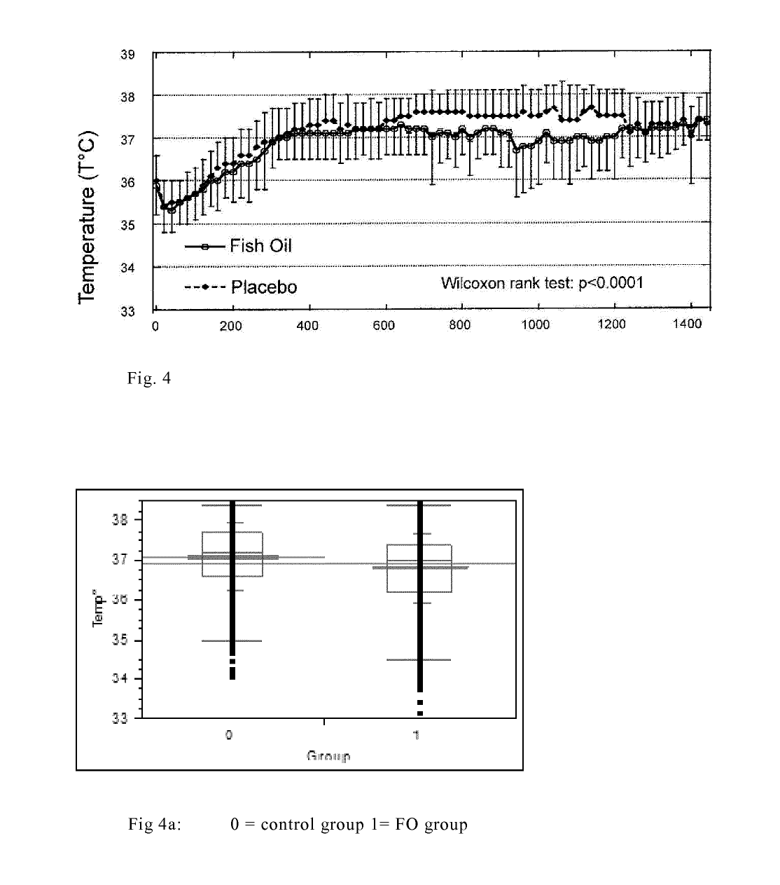

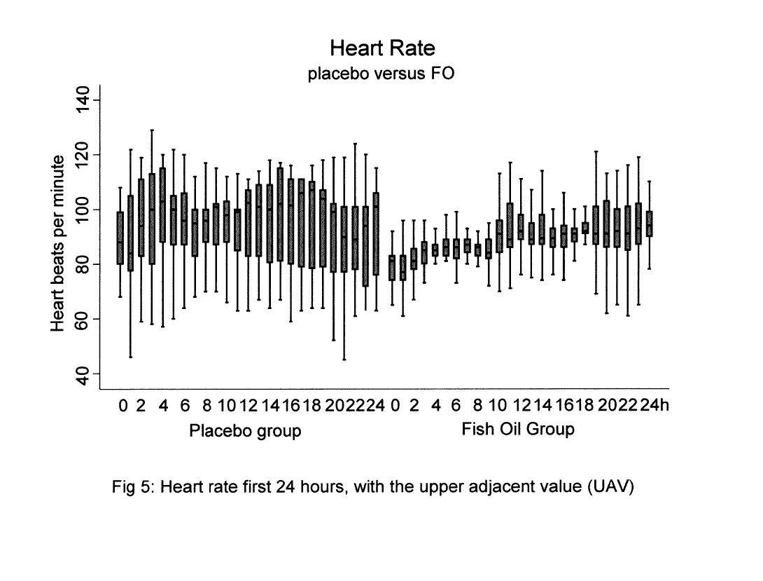

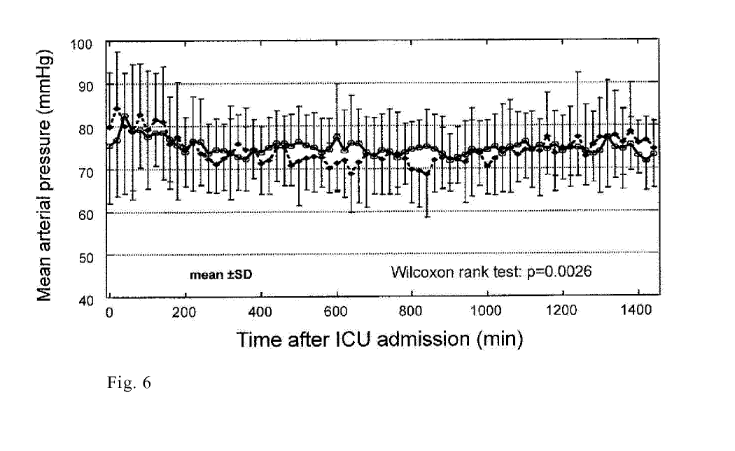

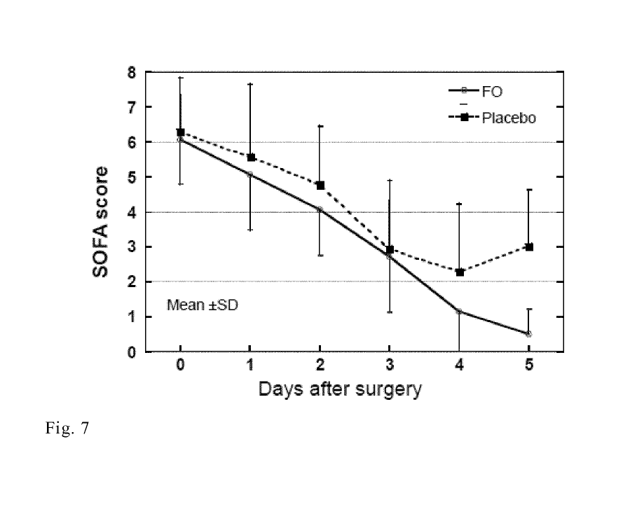

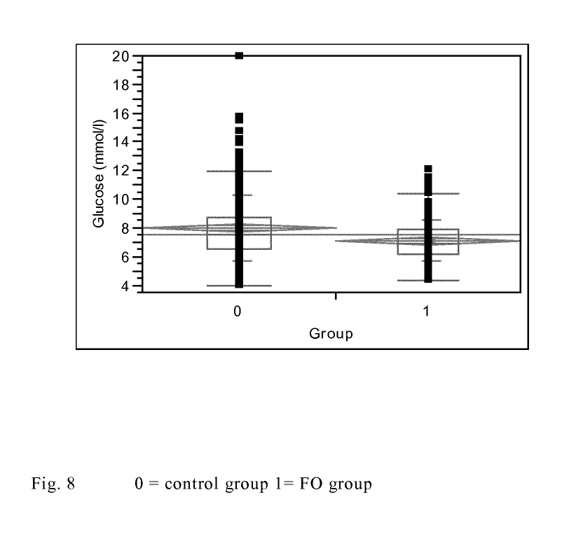

### REFERENCES

1. Kromhout D, Giltay E J, “n-3 fatty acids and cardiovascular events after myocardial infarction” Oeleijnse J M. N Engl J Med. 2010; 363(21):2015-26.
2. A. H. Petter, R L Chiolero, T Cassina, P G Chassot, X M Muller, J P Revelly. Automatic “respirator/weaning” with adaptive support ventilation: the effect on duration of endrotracheal intubation and patient management. Anesth Analg, 97 (2003), pp. 1743-1750.
3. F. Roques, S. A. Nashef, P Michel et al. Risk factors and outcome in European cardiac surgery: analysis of the EuroSCORE multinational database of 19030 patients. Eur J Cardiothorac Surg, 15 (1999), pp. 816-822; discussion 222-223.
4. A. C. Kajdacsy-Balla Amaral, F. M. Andrade, R Moreno, A Artigas, F Cantraine, J L Vincent. Use of the sequential organ failure assessment score as a severity score. Intensive Care Med, 31 (2005), pp. 243-249.
5. F. Delodder, C. Joseph, P Maravic et al. Tight glucose control managed by ICU nurses induces extremely low rates of hypoglycaemia, Crit Care, 15 (Suppl 1) (2011), pp. P395.
6. J. L. Vincent, R Moreno, J Takala et al. The SOFA (Sepsis-related Organ Failure Assessment) score to describe organ dysfunction/failure. On behalf of the Working Group on Sepsis-Related Problems of the European Society of Intensive Care Medicine. Intensive Care Med, 22 (1996), pp. 707-710.
7. L. Tappy, M. M. Berger, J M Schwarz, J P Revelly, R Chioléro. Metabolic effects of parenteral nutrition enriched with n-3 polyunsaturated fatty acids in critically ill patients. Clin Nutr, 25 (2006), pp. 588-595.
8. R. Clive Landis, J M Murkin, D A Stump et al. Consensus statement: minimal criteria for reporting the systemic inflammatory response to cardiopulmonary bypass. Heart Surg Forum, 13 (2010), pp. E116-E123.
9. E. O. Owens. Endogenous carbon monoxide production in disease. Clin Biochem, 43 (2010), pp. 1183-1188.
10. P Schober, M Kalmanowicz, L A Schwarte, S A Loer. Cardiopulmonary bypass increases endogenous carbon monoxide production. J Cardiothorac Vase Anesth, 23 (2009), pp. 802-806.
11. P. C. Calder. n-3 fatty acids, inflammation, and immunity—relevance to postsurgical and critically ill patients. Lipids, 39 (2004), pp. 1147-1161.
12. PC Calder. The relationship between the fatty acid composition of immune cells and their function. Prostaglandins Leukot Essent Fatty Acids 79 (2008), pp. 101-108.
13. M. Rusca, M. Oddo, M. D. Schaller, L Liaudet. Carboxyhemoglobin formation as an unexpected side effect of inhaled nitric oxide therapy in severe acute respiratory distress syndrome. Crit Care Med, 32 (2004), pp. 2537-2539.
14. K. A. Hunter, G J Singh, C O Simpkins. Cyclic GMP is a measure of physiologic stress. J Natl Med Assoc, 93 (2001), pp. 256-262.
15. T. Liu, P Korantzopoulos, M Shehata, G Li, X Wang, S Kaul. Prevention of atrial fibrillation with omega-3 fatty acids: a meta-analysis of randomised clinical trials. Heart, 97 (2011), pp. 1034-1040.
16. D. Mozaffarian, A Geelen, I. A Brouwer, J. M Geleijnse, P. L Zock, M. B Katan. Effect of fish oil on heart rate in humans: a meta-analysis of randomized controlled trials. Circulation, 112 (2005), pp. 1945-1652.
17. L. Calo, L. Bianconi, F. Colivicchi et al. N-3 Fatty acids for the prevention of atrial fibrillation after coronary artery bypass surgery: a randomized, controlled trial. J Am Coll Cardiol, 45 (2005), pp. 1723-1728.
18. The Merck Manual, online version, accessed on Aug. 23, 2012 http://www.merckmanuals.com/professional/critical_care_medicine/respirat ory_failure_and_mechanical_ventilation/overview_of mechanical_ventilatio n.html

1. Medicament comprising a therapeutically effective amount of at least one omega-3 polyunsaturated fatty acid for use in the reduction and prevention of post-ischemic damages in patients undergoing elective surgery.

2. Medicament according to claim 1 for use in reduction and prevention of post-ischemic damages associated with reperfusion injuries.

3. Medicament according to claim 1 or 2, wherein the patients are selected from surgery patients wherein the surgery requires substantial induction of ischemia.

4. Medicament according to any of the preceding claims for additional use in prevention and/or treatment of
a) Postoperative respiratory dysfunction,
b) Metabolic instability such as postoperative impairment of glucose homeostasis,
c) Postoperative febrile response related to the post surgical stress response, and/or
d) Postoperative organ dysfunctions as summarized in the SOFA score
which most likely occur due to ischemia-reperfusion injury following major elective surgery.

5. Medicament according to any preceding claim, which is formulated for intravenous injection/infusion.

6. Medicament according to any preceding claim, which is administered peri-operatively, preferably at least on the day before surgery in combination with at least on one of during surgery and the day after.

7. Medicament according to any preceding claim, wherein the administration is
a) at least on one of the day before surgery and/or pre-operatively and
b) intra-operatively and
c) immediately after surgery and/or the day after surgery.

8. Medicament according to any preceding claim, which is administered in one or more doses of 0.01 to 0.5. g omega-3 polyunsaturated fatty acid/kg body weight.

9. Medicament according to any preceding claim, wherein the medicament comprises eicosapentaenoic acid (EPA) and/or docosahexaenoic acid (DHA).

10. Medicament according to claim 8 or 9, wherein each dose is administered over a time period of 10 min-6 hours, preferably 1 hour-4 hours, more preferred 1.5 hours to 2.5 hours.

11. Medicament according to any preceding claim, wherein the composition does not contain amino acids or proteins and carbohydrates.

12. Medicament according to any of the preceding claims wherein the composition further comprises 150-300 mg/l alpha-tocopherol and/or 0.9-4.7 g/l arachidonic acid.

13. Medicament according to any preceding claim, wherein the use is further characterized by an induction of lower peak airway pressure.

14. Medicament according to any preceding claim, preferably claim 13, wherein the use is further characterized by decreasing the sequential organ failure assessment (SOFA) score of a patient.
